(19) United States
(12) Patent Application Publication
(10) Pub. No.: US 2023/0272034 A1
(43) Pub. Date: Aug. 31, 2023

(54) T-CELL RECEPTOR OF HLA-A11-RESTRICTED HEPATITIS B VIRUS HBC141-151 EPITOPE PEPTIDE, AND APPLICATION THEREOF

(71) Applicant: INSTITUTE OF MICROBIOLOGY, CHINESE ACADEMY OF SCIENCES, Beijing (CN)

(72) Inventors: Xuyu ZHOU, Beijing (CN); Xundong WEI, Beijing (CN); Wei XU, Beijing (CN); Jianhua ZHANG, Beijing (CN); Man HUANG, Beijing (CN)

(21) Appl. No.: 17/779,174

(22) PCT Filed: Jul. 28, 2020
(86) PCT No.: PCT/CN2020/105157

§ 371 Date: May 24, 2022

(30) Foreign Application Priority Data
Nov. 26, 2019 (CN) 201911170818.7

(51) Int. Cl. C07K 14/725; A61K 39/00; A61P 31/20; C12N 15/86

(52) U.S. Cl. CPC C07K 14/7051; A61K 39/4632; A61K 39/4611; A61P 31/20; C12N 15/86; C12N 2740/15043

(57) ABSTRACT

The present invention discloses T-cell receptor of HLA-A11-restricted hepatitis B virus HBc 141-151 epitope peptide and applications thereof. The T cell receptor comprises an α chain and β chain; the α chain comprises three complementarity determining regions with amino acid sequences shown in positions 48 to 53, positions 71 to 77 and positions 112 to 121 of SEQ ID NO. 2, respectively; the β chain comprises three complementarity determining regions with amino acid sequences shown in positions 46 to 50, positions 68 to 73 and positions 111 to 122 of SEQ ID NO. 4, respectively. Experiments demonstrated that the T cell receptor exhibits both HBV polypeptide epitope-dependent activation and proliferation ability and also exhibits an ability to kill target cells both in vivo and in vitro.

### RELATED APPLICATIONS

The present application is a U.S. National Phase of International Application Number PCT/CN2020/105157, filed Jul. 28, 2020, and claims priority to Chinese Application Number 201911170818.7, filed Nov. 26, 2019.

### INCORPORATION BY REFERENCE

The sequence listing provided in the file entitled Amended_SQL_2022-09-27.txt, which is an ASCII text file that was created on Sep. 27, 2022, and which comprises 26,329 bytes, is hereby incorporated by reference in its entirety.

### TECHNICAL FIELD

The present invention belongs to the field of biomedicine, and particularly relates to a T cell receptor of HLA-A11-restricted hepatitis B virus HBc₁₄₁₋₁₅₁ epitope peptide and its application.

### BACKGROUND OF THE INVENTION

Hepatocellular carcinoma (HCC), the most common type of primary liver cancer, has insidious clinical manifestations and typically lacks symptoms in the early stages. Although some progress has been made in the diagnosis and treatment of liver cancer in recent years, the prognosis for patients with liver cancer is still poor and the five-year survival rate is extremely low. The reasons for this poor prognosis are two-fold in that 1) HCC is not sensitive to current chemotherapeutic drugs so that most HCC patients lack access to effective treatment methods and 2) HCC is often diagnosed at an advanced stage of the disease which precludes local ablation methods which would otherwise improve the patient's condition. At present, surgical resection and liver transplantation are still the most effective methods for the treatment of HCC. However, when HCC develops to a certain stage, tumor cells may metastasize to other organs (such as lungs, bones, brain, etc.). Before liver transplantation, the current examination methods cannot accurately detect the metastasized cells. After surgery, however, due to the immunosuppressive state induced to prevent rejection of the transplanted liver, latent micro-lesions in other organs may lead to the recurrence of HCC and the postoperative survival of such patients is not particularly good. Therefore, there is an urgent need to find new and effective adjuvant therapies.

Immune cell therapy is the most rapidly developing field in biomedicine. In 2013, the journal Science Translational Medicine predicted that cell therapy would become “the third pillar of future medicine.” Since October 2017, the U.S. Food and Drug Administration (FDA) has approved the CAR-T drugs from Novartis and KITE, creating a new era of immune cell therapeutic drugs and showing broad application prospects in clinical treatment. In the field of immune cell therapy for solid tumors, ideal target antigens are tumor-specific antigens that are expressed only on the surface of tumor cells. Unfortunately, most of the antigens expressed by tumors are not tumor-specific, so most CAR-T and TCR-T target tumor-associated antigens, which often leads to the possibility of “off-target” impact. At present, some tumor-associated antigens, such as α-fetoprotein, NY-ESO, and MAGE, which are widely used at present despite these limitations.

Among many environmental risk factors related to HCC, hepatitis B virus (HBV) and hepatitis C virus (HCV) infections are directly related to the occurrence of HCC. Up to 80% of HCC is attributable to HBV or HCV infection. In China, HBV infection is the main cause, and 90% of HCC patients are HBsAg positive. HBV infection in the human body can stimulate the body to produce a series of humoral and cellular immune responses, which can usually clear the virus in infected cells and allow the body to recover. However, if the body's immune response is low and insufficient to clear the virus, the virus will persist and develop into chronic hepatitis B. Chronic hepatitis B is an important risk factor for the occurrence of liver cirrhosis and HCC. Chronic hepatitis B mainly involves deficiencies in the antiviral immune response at various stages of the immune response, especially the decrease in the number and function of HBV-specific CD8+ T cells, which leads to immune tolerance accompanied by immune-mediated inflammatory liver damage.

Currently, there is no drug that can cure hepatitis B. The clinical treatment for chronic hepatitis B mainly adopts antiviral drugs, including interferons (IFNs), nucleoside and nucleotide drugs, etc., which exert antiviral effects by immunomodulating or interfering with HBV replication. However, these treatments cannot completely clear the virus, making patients prone to drug resistance, virus mutation, and recurrence of the disease, etc.

HBV-specific CD8+ T cells play a decisive role in controlling viral replication, viral clearance, and clinical recovery from HBV infection. Adoptive transfer of HBV-specific TCR gene-modified T cells (TCR-T) has initially demonstrated very good antiviral activity. In the field of HCC, HBV can also serve as a unique tumor-associated antigen. Although known HBV-HCC hepatoma cells do not express complete HBV antigens, in the natural history of chronic hepatitis B infection, the virus often integrates itself into the human genome, eventually forming HCC cells that carry these HBV genomes. Singaporean scholars have found that although HBV-HCC hepatoma cells do not express complete HBV antigens, they contain short fragments of HBV mRNAs, which can encode epitope polypeptides that can be recognized and activated by HBV-specific T cells. They enrolled 2 liver transplant patients with HCC recurrence and pulmonary metastatic tumors, selected HBV-specific T cell receptors (TCRs) according to the expression of HBV mRNA in the tumors, used genetic engineering to express these TCRs in autologous T cells, and then adoptively transferred the cells to the patients. These TCR-T cells did not affect liver function, and in one patient, ⅚ of the pulmonary metastatic tumors decreased in size within 1 year. These results suggest that in the treatment of HCC patients, especially liver transplant patients, T cell adoptive immunotherapy using HBV epitopes on hepatoma cells as tumor-associated antigens may clear HBV-HCC hepatoma cells. This HCC-specific marker means that adverse effects outside the tumor are largely suppressed, with little or no involvement of other organs. In addition, other clinical research teams have used TCR-T cells that specifically recognize the HLA-A2-restricted epitope HBs183-191 of HBV to treat HCC metastasis in liver transplant patients by inducing TCR-redirected T cells in vitro. This work has demonstrated that these TCR-redirected T cells can be used for actively targeting specific hepatoma cells and show great application potential and value for improved treatments.

The current reported research on therapeutic T cells is mainly limited to the HLA-A2 population, while the attention to other HLA populations has been limited. The HLA-A3 superfamily (including HLA-A11, HLA-A33, HLA-A68, HLA-A31, etc.) occupies the largest proportion (about 52.7%) in HLA typing of Chinese population. One of the distinguishing features of HLA-A3 superfamily members is that different members share a common polypeptide binding property, that is, they prefer to bind to polypeptide epitopes with basic amino acids at the C-terminus. Therefore, the research on the treatment of HCC for the HLA-A3 superfamily-restricted population will have good popularity and generality. Among them, HLA-A11 is the most widely distributed in the A3 superfamily, and statistical analysis also shows that the HLA-A11 gene frequency is the highest in Chinese hepatitis B patients.

HLA transgenic animal models have played an important role in preclinical trials and basic experimental research. HLA-A11 transgenic mice have been reported before. However, additional data has shown that HLA-A11 molecules preferentially bind to polypeptide epitopes with basic amino acids at the C-terminus, which are inconsistent with the peptide binding preferences of the transporter associated protein (TAP) in the mouse self-antigen presentation system, and thus HLA-A11 transgenic mice are significantly defective in the presentation of HLA-A11-restricted epitopes.

BAC transgenic mice containing human TAP-LMP gene cluster (hTAP-LMP transgenic mice) were crossed with HLA-A11 transgenic mice to obtain HLA-A11/hTAP-LMP transgenic mice. Compared with HLA-A11 transgenic mice, HLA-A11/hTAP-LMP transgenic mice have a greater expression of HLA-A11-restricted CTL epitopes.

In conclusion, immune cell technology for the treatment of hepatitis B and HCC has entered the clinical trial stage and has made progress, showing broad application prospects. However, in-depth research is urgently needed for the treatment of HLA-A11-restricted HCC. Particularly in the affected Chinese population.

### SUMMARY OF THE INVENTION

The purpose of the present invention is to prepare a medicament for preventing and/or treating diseases caused by HBV infection.

The present invention firstly protects a T cell receptor that recognizes HLA-A11-restricted hepatitis B virus HBc141-151 epitope peptide, which comprises an α chain and a β chain. The α chain can comprise three complementarity determining regions with amino acid sequences shown in positions 48 to 53, positions 71 to 77 and positions 112 to 121 of SEQ ID NO. 2, respectively; or variants of these sequences with up to 3, 2 or 1 amino acid change. The β chain can comprise three complementarity determining regions with amino acid sequences shown in positions 46 to 50, positions 68 to 73 and positions 111 to 122 of SEQ ID NO. 4, respectively; or variants of these sequences with up to 3 amino acid changes.

In the T cell receptor, the amino acid sequence of the variable region of the α chain can be shown in positions 22 to 112 of SEQ ID NO. 2; or variants of these sequences with up to 3, 2 or 1 amino acid change. The amino acid sequence of the variable region of the β chain can be shown in positions 20 to 113 of SEQ ID NO. 4; or variants of these sequences with up to 3 amino acid change.

The amino acid sequence of the constant region of the α chain is shown in positions 133 to 268 of SEQ ID NO. 2.

The amino acid sequence of the constant region of the β chain is shown in positions 133 to 305 of SEQ ID NO. 4.

In the T cell receptor, the amino acid sequence of the α chain can be shown in SEQ ID NO. 2. The amino acid sequence of the β chain can be shown in SEQ ID NO. 4.

A nucleic acid molecule coding for any one of the above-mentioned T cell receptors is also considered to be within the scope of the present invention.

The nucleic acid molecule coding for any one of the above-mentioned T cell receptors can comprise a nucleic acid molecule coding for the α chain of the T cell receptor and a nucleic acid molecule coding for the β chain of the T cell receptor.

The nucleotide sequences coding for the three complementarity determining regions in the α chain of the T cell receptor can be shown in positions 142 to 159, positions 211 to 231 and positions 334 to 363 of SEQ ID NO. 1, respectively; or the sequences which have at least 99%, at least 95%, at least 90%, at least 85%, or at least 80% identity to these sequences and encode the same amino acid residues.

The nucleotide sequences coding for the three complementarity determining regions in the β chain of the T cell receptor can be shown in positions 136 to 150, positions 202 to 219 and positions 331 to 366 of SEQ ID NO. 3, respectively; or the sequences which have at least 99%, at least 95%, at least 90%, at least 85%, or at least 80% identity to these sequences and encode the same amino acid residues.

The nucleotide sequence coding for the variable region of the α chain can be shown in positions 64 to 336 of SEQ ID NO. 1; or a sequence which has at least 99%, at least 95%, at least 90%, at least 85%, or at least 80% identity to this sequence and encodes the same amino acid residues.

The nucleotide sequence coding for the variable region of the β chain is shown in positions 58 to 339 of SEQ ID NO. 3; or a sequence which has at least 99%, at least 95%, at least 90%, at least 85%, or at least 80% identity to this sequence and encodes the same amino acid residues.

The nucleotide sequence of the constant region of the α chain is shown in positions 397 to 807 of SEQ ID NO. 1.

The nucleotide sequence of the constant region of the β chain is shown in positions 397 to 918 of SEQ ID NO. 3.

The nucleotide sequence of the nucleic acid molecule coding for the α chain is shown in SEQ ID NO. 1.

The nucleotide sequence of the nucleic acid molecule coding for the β chain is shown in SEQ ID NO. 3.

An expression cassette, a vector or a cell comprising any one of the above-mentioned nucleic acid molecules also is also considered to be within the scope of the present invention.

The cell can be a Jurkat cell or a T cell. The T cell can be a human T cell or a mouse T cell.

The vector can be a retroviral vector or a lentiviral vector.

The retroviral vector can be a recombinant plasmid obtained by inserting the nucleic acid molecule coding for the α chain of the T cell receptor and the nucleic acid molecule coding for the 3 chain of the T cell receptor between the multiple cloning sites (such as between the XhoI and EcoRI restriction sites) of the retroviral vector MSCV-IRES-GFP.

The retroviral vector can be a recombinant plasmid obtained by inserting a DNA sequence between the multiple cloning sites (such as between the XhoI and EcoRI restriction sites) of the retroviral vector MSCV-IRES-GFP; the DNA sequence is obtained by linking the nucleic acid molecule coding for the α chain and the nucleic acid molecule coding for the 3 chain with the sequence coding for a connecting peptide (such as T2A self-cleaving polypeptide).

In one embodiment of the present invention, the retroviral vector can be a recombinant plasmid obtained by replacing the small DNA fragment between the XhoI and EcoRI restriction sites of the retroviral vector MSCV-IRES-GFP with a DNA molecule having the nucleotide sequence shown in SEQ ID NO. 5. In SEQ ID NO. 5, the sequence shown in positions 1 to 804 is the complete gene coding for the α chain, the sequence shown in positions 805 to 867 is the gene coding for the T2A self-cleaving polypeptide, and the sequence shown in positions 868 to 1785 is the complete gene coding for the β chain.

The above-mentioned retroviral vector MSCV-IRES-GFP is a recombinant plasmid obtained by replacing the small DNA fragment between the XhoI and ClaI restriction sites of the MO vector with an IRES nucleotide sequence (Genbank: MG550106.1) and a fluorescent marker protein GFP nucleotide sequence (Genbank: MH777595.1).

The lentiviral vector can be a recombinant plasmid obtained by inserting the nucleic acid molecule coding for the α chain of the T cell receptor and the nucleic acid molecule coding for the β chain of the T cell receptor between the multiple cloning sites (such as between the EcoRI and BamHI restriction sites) of the lentiviral packaging vector pCDH-MSCV-MCS-IRES-GFP (System Biosciences, No.: CD731B-1).

The lentiviral vector can be a recombinant plasmid obtained by inserting a DNA sequence between the multiple cloning sites (such as between the EcoRI and BamHI restriction sites) of the lentiviral packaging vector pCDH-MSCV-MCS-IRES-GFP; the DNA sequence is obtained by linking the nucleic acid molecule coding for the α chain and the nucleic acid molecule coding for the R chain with the sequence coding for a connecting peptide (such as T2A self-cleaving polypeptide).

In one embodiment of the present invention, the lentiviral vector can be a recombinant plasmid obtained by replacing the small DNA fragment between the EcoRI and BamHI restriction sites of the lentiviral packaging vector pCDH-MSCV-MCS-IRES-GFP with a DNA molecule having the nucleotide sequence shown in SEQ ID NO. 5. In SEQ ID NO. 5, the sequence shown in positions 1 to 804 is the complete gene coding for the α chain, the sequence shown in positions 805 to 867 is the gene coding for the T2A self-cleaving polypeptide, and the sequence shown in positions 868 to 1785 is the complete gene coding for the β chain.

A T cell having any one of the above-mentioned T cell receptors is also considered to be within the scope of the present invention.

A pharmaceutical composition comprising the T cell having any one of the above-mentioned T cell receptors or “any one of the above-mentioned expression cassettes, vectors or cells” is also considered to be within the scope of the present invention. The pharmaceutical composition can be used to prevent and/or treat diseases caused by HBV infection.

The scope of the present invention also encompasses use of any one of the above-mentioned T cell receptors, or any one of the above-mentioned nucleic acid molecules, or any one of the above-mentioned vectors or cells, or any one of the T cells having the above-mentioned T cell receptor for at least one of the A1)-A4) uses:
A1) preparing a medicament for preventing and/or treating diseases caused by HBV infection;
A2) preventing and/or treating diseases caused by HBV infection;
A3) killing a target cell in vivo or in vitro;
A4) clearing chronic HBV infection.

In the above uses, the target cell can be a spleen cell or a PBMC cell. The spleen cell can be a spleen cell loaded with polypeptide HBc141-151. The PBMC cell can be a PBMC cell loaded with polypeptide HBc141-151. The spleen cell loaded with polypeptide HBc141-151 can be a spleen cell loaded with polypeptide HBc141-151 in mice (e.g., HLA-A11 transgenic mice). The PBMC cell loaded with polypeptide HBc141-151 can be a PBMC cell loaded with polypeptide HBc141-151 in mice (e.g., HLA-A11 transgenic mice) or an HLA-A11+ PBMC cell loaded with polypeptide HBc141-151. The HLA-A11+ PBMC cell refers to a PBMC cell of HLA-A11-positive healthy people.

The scope of the present invention also encompasses methods for preventing and/or treating diseases caused by HBV infection, which can comprise the step of using the above-mentioned T cell receptor, or the nucleic acid molecule, or “the expression cassette, vector or cell,” or the above-mentioned T cell having any one of the above-mentioned T cell receptors to prevent and/or treat diseases caused by HBV infection including, for example, chronic hepatitis B or hepatocellular carcinoma.

Over the course of conducting a large number of experiments, the inventors of the present invention have isolated and identified a pair of HBV-specific TCR sequences, successfully constructed transgenic mice expressing this pair of TCRs, and verified in vitro that TCR transgene-positive CD8 cells (i.e., TCR-T cells) have HBV polypeptide epitope-dependent activation and proliferation ability. Using animal in vivo and in vitro killing target cell experiments, the inventors proved that this pair of TCRs exhibits good effectiveness in killing target cells (the spleen cells or PBMC cells loaded with polypeptide HBc141-151 in HLA-A11 transgenic mice) and verified in vitro that human TCR-T cells also have the ability to specifically kill target cells (HLA-A11-restricted human PBMC cells loaded with polypeptide HBc141-151). In addition, the inventors' animal experiments suggest that this pair of TCR sequences may be an effective method for clearing HBV-infected cells. The T cell receptor of HLA-A11-restricted HBC141-151 epitope peptide provided by the present invention represents a significant advancement of the art.

### DESCRIPTION OF THE DRAWINGS

FIG. 1 is a schematic flow chart of screening HLA-A11-restricted HBV-specific TCR sequences.

FIG. 2 shows the staining results of Jurkat cells expressing TCR.

FIG. 3 shows the identification of TCR transgenic mice.

FIG. 4 shows that the CD8+ T cells (i.e., TCR-T cells) of TCR transgenic mice have both HBV polypeptide-dependent activation and proliferation ability.

FIG. 5 shows that TCR-T cells are effective in killing target cells in vitro.

FIG. 6 shows that TCR-T cells are effective in killing target cells in vivo.

FIGS. 7A and 7B show that TCR-T cells can effectively clear chronic HBV infection in vivo.

FIGS. 8A and 8B show that human TCR-T cells are effective in killing target cells in vitro.

### DETAILED DESCRIPTION OF THE INVENTION

The following examples facilitate a better understanding of the present invention, but do not limit the present invention.

The experimental methods utilizes in the following examples are conventional methods unless otherwise specified.

The test materials used in the following examples are commercially available unless otherwise specified.

The quantitative tests in the following examples were conducted in triplicate, and the results were averaged.

HLA-A11/hTAP-LMP transgenic mice, polypeptide HBc123-157, helper polypeptide HBc128-140 and polypeptide HBc141-151 are described in the report by Man Huang, Wei Zhang, Jie Guo, Xundong Wei, Krung Phiwpan, Jianhua Zhang, and Xuyu Zhou, Improved Transgenic Mouse Model for Studying HLA Class I Antigen Presentation, Scientific Report, doi:10.1038/srep33612 (2016).

In the following examples, the hydrodynamic tail vein injection method used was described in “Liu F, Song Y, and Liu D., Hydrodynamics-based Transfection in Animals by Systemic Administration of Plasmid DNA, Gene Therapy, doi: 10.1038/sj.gt.3300947 (1999).

In the following examples, the pAAV/HBV1.2 plasmid used was described in Huang, L. R., Wu, H. L., Chen, P. J., and Chen, D. S., An immunocompetent mouse model for the tolerance of human chronic hepatitis B virus infection, PNAS USA, 103.17862-17867, doi: 10.1073/pnas.0608578103 (2006).

10×PCR buffer, dNTP mix, and Taq DNA polymerase were obtained from TAKARA Biomedical Technology Co., Ltd.

C57BL/6 mice and ICR mice were obtained from Beijing HFK Bioscience Co., Ltd.

B6D2F1 mice were obtained from Beijing Vital River Laboratory Animal Technology Co., Ltd.

### Example 1. Acquisition and Application of TCR-T Cells

I. Screening of all-Restricted HBV-Specific TCR Sequences Using HLA-A11/hTAP-LMP Transgenic Mice.

1. 100 μL of PBS buffer containing 100 μg of polypeptide HBc123-157 and 100 g of helper polypeptide HBc128-140 was mixed with 100 μL of IFA, emulsified, and then subcutaneously injected into HLA-A11/hTAP-LMP transgenic mice at multiple points. A primary CTL immune response against the HBc141-151 epitope was induced in HLA-A11/hTAP-LMP transgenic mice.

2. On the 14th day after the completion of step 1, the pAAV/HBV1.2 plasmid was injected into the tail vein of HLA-A11/hTAP-LMP transgenic mice at a dose of 10 μg per mouse using the hydrodynamic tail vein injection method.

The pAAV/HBV1.2 plasmid contains 1.2 copies of the genome of HBV virus and can transiently transfect mouse hepatocytes and express hepatitis B virus antigens and produce virus particles by the hydrodynamic tail vein injection method. Therefore, the plasmid can be used to simulate the early HBV infection in mice. The purpose of this step is to induce a secondary CTL immune response against HBc141-151 after early HBV infection, and to generate a large number of HBc141-151 antigen-specific CD8+ T cells.

3. On the 8th day after the completion of step 2, HLA-A11/hTAP-LMP transgenic mice were sacrificed and peripheral blood mononuclear cells were collected for Tetramer staining, and then HLA-A11/HBc141-151 Tetramer (NIH Tetramer Facility) and CD8 double-positive T cells were sorted by flow cytometry. The antigen-specific CTLs were manually aspirated under the microscope using a mouth-aspirating microinjection needle to obtain single antigen-specific CTL cells.

Random primers were obtained from Sangon Biotech (Shanghai) Co., Ltd.

2× buffer and RT enzymes were obtained from Beijing Transgen Biotech Co., Ltd.

4. After the completion of step 3, the reverse transcription system Mix1 (composed of 0.5 μL of Random primer and 4.5 μL of DEPC-treated H₂O) and the reverse transcription system Mix2 (composed of 5 μL of 2× buffer and 0.5 μL of RT enzyme) were prepared. Then under the microscope, a microinjection needle was used to aspirate a single antigen-specific CTL cell into the reverse transcription system Mix1, and the system was placed in a water bath at 70° C. for 5 min and then in an ice bath for 2 min; then the reverse transcription system Mix2 was added, and PCR was performed at 25° C. for 5 min, 42° C. for 30 min and 85° C. for 5 min to synthesize single-cell cDNA.

5. After the completion of step 4, two rounds of PCR were performed using TCR-specific degenerate primers.

The first round PCR: TCRα-mix, 23 primers in V region and 1 primer in C region; TCRβ-mix, 19 primers in V region and 1 primer in C region.

The second round PCR: TCRα-mix (in), 23 primers in V region and 1 primer in C region; TCRβ-mix (in), 19 primers in V region and 1 primer in C region.

The nucleotide sequences of the primers are shown in TABLE 1.

TABLE 1
Primers for TCR-α

| | Forward primer in the | Forward primer in the |
|---|---|---|
| Primer name | first round | second round |
| TRAV1 | GGTTATCCTGGTACCAGCA | CTCCACATTCCTGAGCC |
| | (SEQ ID NO. 6) | (SEQ ID NO. 50) |
| TRAV2 | CATCTACTGGTACCGACAGG | ACTCTGAGCCTGCCCT |
| | (SEQ ID NO. 7) | (SEQ ID NO. 51) |
| TRAV3 | GGCGAGCAGGTGGAG | GCCCTCCTCACCTGAG |
| | (SEQ ID NO. 8) | (SEQ ID NO. 52) |
| TRAV4 | TCTGSTCTGAGATGCAATTTT | GGITIMAGGAACAAAGGAGAA |
| | (SEQ ID NO. 9) | T(SEQ ID NO. 53) |
| TRAV5-1/5-4 | GGCTACTTCCCTTGGTATAAGC | ATYCGTTCAAATATGGAAAGA |
| (D) | AAGA (SEQ ID NO. 10) | AA (SEQ ID NO. 54) |
| TRAV6-1/6-2 | CAGATGCAAGGTCAAGTGAC | GGAGAAGGTCCACAGCTC |
| | (SEQ ID NO. 11) | (SEQ ID NO. 55) |
| TRAV6-3/6-4 | AAGGTCCACAGCTCCTTC | CAACTGCCAACAACAAGG |
| (D) | (SEQ ID NO. 12) | (SEQ ID NO. 56) |
| TRAV6-5/6-7 | GTTCTGGTATGTGCAGTATCC | TCCTTCCACTTGCAGAAAG(SE |
| (D) | (SEQ ID NO. 13) | Q ID NO. 57) |
| TRAV6-6 | AGATTCCGTGACTCAAACAG | ACGGCTGGCCAGAAG |
| | (SEQ ID NO. 14) | (SEQ ID NO. 58) |
| TRAV7 | AGAAGGTRCAGCAGAGCCCAG | CAKGRCYTCYYTCAACTGCAC |
| | AATC (SEQ ID NO. 15) | (SEQ ID NO. 59) |
| TRAV8 | GAGCRTCCASGAGGGTG | AGAGCCACCCTTGACAC |
| | (SEQ ID NO. 16) | (SEQ ID NO. 60) |
| TRAV9 | CCAGTGGTTCAAGGAGTG | GCTTYGAGGCTGAGTTCAG |
| | (SEQ ID NO. 17) | (SEQ ID NO. 61) |
| TRAV10/10a | AGAGAAGGTCGAGCAACAC | CTACACTGAGTGTTCGAGAGG |
| (D) | (SEQ ID NO. 18) | (SEQ ID NO. 62) |
| TRAV11 | AAGACCCAAGTGGAGCAG | AACAGGACACAGGCAAAG |
| | (SEQ ID NO. 19) | (SEQ ID NO. 63) |
| TRAV12 | TGACCCAGACAGAAGGC | GGTTCCACGCCACTC |
| | (SEQ ID NO. 20) | (SEQ ID NO. 64) |
| TRAV13 | TCCTTGGTTCTGCAGG | TGCAGGAGGGGGAGA |
| | (SEQ ID NO. 21) | (SEQ ID NO. 65) |
| TRAV14 | GCAGCAGGTGAGACAAAG | CTCTGACAGTCTGGGAAGG |
| | (SEQ ID NO. 22) | (SEQ ID NO. 66) |
| TRAV15 | CASCTTYTTAGTGGAGAGATGG | AYTCTGTAGTCTTCCAGAAAT |
| | (SEQ ID NO. 23) | CAC (SEQ ID NO. 67) |
| TRAV16 | GTACAAGCAAACAGCAAGTG | ATTATTCTCTGAACTTTCAGAA |
| | (SEQ ID NO. 24) | GC (SEQ ID NO. 68) |
| TRAV17 | CAGTCCGTGGACCAGC | TATGAAGGAGCCTCCCTG |
| | (SEQ ID NO. 25) | (SEQ ID NO. 69) |
| TRAV18 | AACGGCTGGAGCAGAG | CAAGATTTCACCGCACG |
| | (SEQ ID NO. 26) | (SEQ ID NO. 70) |
| TRAV19 | GCAAGTTAAACAAAGCTCTCC | GCTGACTGTTCAAGAGGGA |
| | (SEQ ID NO. 27) | (SEQ ID NO. 71) |
| TRAV21 | GTGCACTTGCCTTGTAGC | AATAGTATGGCTTTCCTGGC |
| | (SEQ ID NO. 28) | (SEQ ID NO. 72) |
| | Reverse primer in the | Reverse primer in the |
| | first round | second round |
| TRAC-Rev | GGCATCACAGGGAACG | GCACATTGATTTGGGAGTC |
| | (SEQ ID NO. 29) | (SEQ ID NO. 73) |

Primers for TCR-β

| | Forward primer in the | Forward primer in the |
|---|---|---|
| Primer name | first round | second round |
| TRBV1 | TACCACGTGGTCAAGCTG | GTATCCCTGGATGAGCTG |
| | (SEQ ID NO. 30) | (SEQ ID NO. 74) |
| TRBV 2 | CAGTATCTAGGCCACAATGC | GGACAATCAGACTGCCTC |
| | (SEQ ID NO. 31) | (SEQ ID NO. 75) |
| TRBV3 | CCCAAAGTCTTACAGATCCC | GATATGGGGCAGATGGTG |
| | (SEQ ID NO. 32) | (SEQ ID NO. 76) |
| TRBV4 | GACGGCTGTTTTCCAGAC | CAGGTGGGAAATGAAGTG |
| | (SEQ ID NO. 33) | (SEQ ID NO. 77) |
| TRBV5 | GGTATAAACAGAGCGCTGAG | GCCAGAGCTCATGTTTCTC |
| | (SEQ ID NO. 34) | (SEQ ID NO. 78) |
| TRBV12 | GGGGTTGTCCAGTCTCC | CCAGCAGATTCTCAGTCC |
| | (SEQ ID NO. 35) | (SEQ ID NO. 79) |
| TRBV13 | GCTGCAGTCACCCAAAG | GTACTGGTATCGGCAGGAC |
| | (SEQ ID NO. 36) | (SEQ ID NO. 80) |
| TRBV14 | GCAGTCCTACAGGAAGGG | GGTATCAGCAGCCCAGAG |
| | (SEQ ID NO. 37) | (SEQ ID NO. 81) |
| TRBV15 | GAGTTACCCAGACACCCAG | GTGTGAGCCAGTTTCAGG |
| | (SEQ ID NO. 38) | (SEQ ID NO. 82) |
| TRBV16 | CCTAGGCACAAGGTGACAG | GAAGCAACTCTGTGGTGTG |
| | (SEQ ID NO. 39) | (SEQ ID NO. 83) |
| TRBV17 | GAAGCCAAACCAAGCAC | GAACAGGGAAGCTGACAC |
| | (SEQ ID NO. 40) | (SEQ ID NO. 84) |
| TRBV19 | GATTGGTCAGGAAGGGC | GGTACCGACAGGATTCAG |
| | (SEQ ID NO. 41) | (SEQ ID NO. 85) |
| TRBV20 | GGATGGAGTGTCAAGCTG | GCTTGGTATCGTCAATCG |
| | (SEQ ID NO. 42) | (SEQ ID NO. 86) |
| TRBV23 | CTGCAGTTACACAGAAGCC | GCCAGGAAGCAGAGATG |
| | (SEQ ID NO. 43) | (SEQ ID NO. 87) |
| TRBV24 | CAGACTCCACGATACCTGG | GCACACTGCCTTTTACTGG |
| | (SEQ ID NO. 44) | (SEQ ID NO. 88) |
| TRBV26 | GGTGAAAGGGCAAGGAC | GAGGTGTATCCCTGAAAAGG |
| | (SEQ ID NO. 45) | (SEQ ID NO. 89) |
| TRBV29 | GCTGGAATGTGGACAGG | GTACTGGTATCGACAAGACCC |
| | (SEQ ID NO. 46) | (SEQ ID NO. 90) |
| TRBV30 | CCTCCTCTACCAAAAGCC | GGACATCTGTCAAAGTGGC |
| | (SEQ ID NO. 47) | (SEQ ID NO. 91) |
| TRBV31 | CTAACCTCTACTGGTACTGGCA | CTGTTGGCCAGGTAGAGTC |
| | G (SEQ ID NO. 48) | (SEQ ID NO. 92) |
| | Reverse primer in the | Reverse primer in the |
| | first round | second round |
| TRBC-Rev | CCAGAAGGTAGCAGAGACCC | GGGTAGCCTTTTGTTTGTTTG |
| | (SEQ ID NO. 49) | (SEQ ID NO. 93) |

The first round PCR reaction system was 25 μL, see TABLE 2 for details. The reaction program was as follows: 95° C. for 5 min; 34 cycles of (95° C. for 20 s, 56° C. for 20 s, 72° C. for 45 s); 72° C. for 7 min.

TABLE 2

| | TCR |
|---|---|
| cDNA | 2.5 μL |
| 10×PCR buffer | 2.5 μL |
| 10 mM dNTP mix | 0.5 μL |
| Primer Mix (5 μM) | 0.5 L (final concentration, |
| | 0.1 μM) |
| Taq DNA Polymerase | 0.15 μL (0.75 U) |
| H₂O | 18.85 μL |

The second round PCR reaction system was 25 μL, see TABLE 3 for details. The reaction program was as follows: 95° C. for 5 min; 34 cycles of (95° C. for 20 s, 56° C. for 20 s, 72° C. for 45 s); 72° C. for 7 min.

TABLE 3

| | TCR |
|---|---|
| First round PCR product | 1 μL (diluted gradiently) |
| 10×PCR buffer | 2.5 μL |
| 10 mM dNTP mix | 0.5 μL |
| Primer Mix (5 μM) | 0.5 L (final concentration, |
| | 0.1 μM) |
| Taq DNA Polymerase | 0.15 μL (0.75 U) |
| H₂O | 20.35 μL |

6. After the completion of step 5, the paired TCR-α and TCR-β variable region sequences were obtained after two rounds of PCR. The PCR amplification products were recovered with a gel extraction kit and sequenced. The sequencing results were analyzed on the IMGT website:

http://www.imgt.org/IMGT_vquest/vquest?livret=0&Option=mouseTcR to obtain the α-chain and β-chain sequences of single cells.

A total of 40 antigen-specific CTLs were detected. Among them, from 5 antigen-specific CTLs, α chain sequences and β chain sequences, i.e., 5 pairs of TCR receptor sequences, were obtained. After analysis, the α chain and β chain among these five antigen-specific CTLs were identical.

The gene coding for the variable region of the α chain is shown in positions 64 to 336 of SEQ ID NO. 1; wherein, in SEQ ID NO. 1, the sequences shown in positions 142 to 159, positions 211 to 231 and positions 334 to 363 codes for three CDRs, respectively.

The amino acid sequence of the variable region of the α chain is shown in positions 22 to 112 of SEQ ID NO. 2; wherein, in SEQ ID NO. 2, the sequences shown in positions 48 to 53, positions 71 to 77 and positions 112 to 121 are three complementarity determining regions, respectively.

The gene coding for the variable region of the β chain is shown in positions 58 to 339 of SEQ ID NO. 3; wherein, in SEQ ID NO. 3, the sequences shown in positions 136 to 150, positions 202 to 219 and positions 331 to 366 codes for three CDRs, respectively.

The amino acid sequence of the variable region of the β chain is shown in positions 20 to 113 of SEQ ID NO. 4; wherein, in SEQ ID NO. 4, the sequences shown in positions 46 to 50, positions 68 to 73 and positions 111 to 122 are three complementarity determining regions, respectively.

The TCR receptor composed of this pair of α-chain sequence and 3-chain sequence may have high affinity for the HLA-A11-restricted CTL epitope HBc141-151 and were derived from the same T-cell clone.

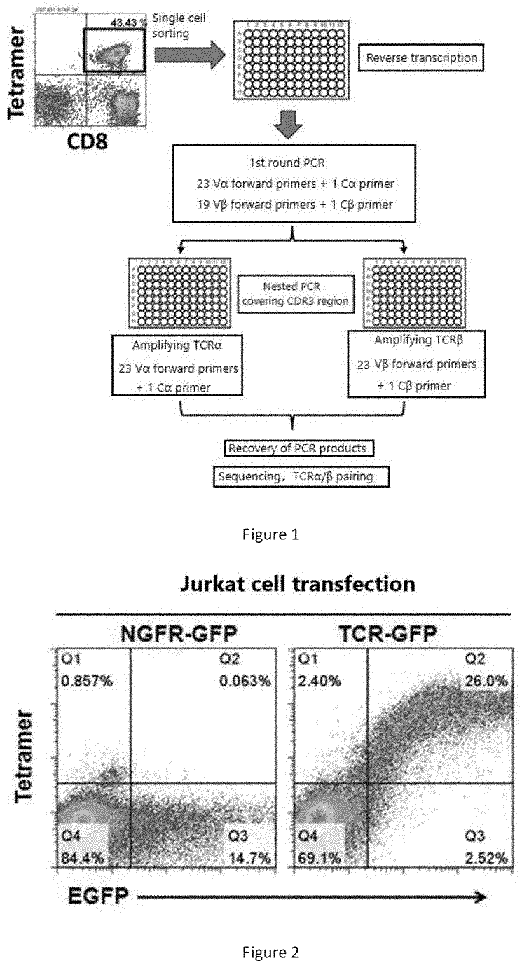

The experimental flow chart of steps 3 to 6 is shown in FIG. 1.

II. Acquisition and Identification of Jurkat Cells Expressing TCR

1. According to the variable region sequences of the α chain and the R chain obtained in step 1 and referring to the constant region sequences of the α chain and β chain of the mouse genome on NCBI, the complete genes coding for the α chain and the β chain of the TCR receptor specific for HLA-A11-restricted hepatitis B virus HBc141-151 were obtained and synthesized artificially.

The complete gene coding for the α chain is shown in SEQ ID NO. 1, which encodes the α chain shown in SEQ ID NO. 2.

The complete gene coding for the β chain is shown in SEQ ID NO. 3, which encodes the β chain shown in SEQ ID NO. 4.

2. The small DNA fragment between the XhoI and EcoRI restriction sites of the retroviral vector MSCV-IRES-GFP was replaced with the DNA molecule shown in SEQ ID NO. 5, and the rest sequence remained unchanged to obtain the recombinant plasmid MSCV-TCR-IRES-GFP (i.e., recombinant plasmid MSCV-TCR-GFP). In SEQ ID NO. 5, the sequence shown in positions 1 to 804 is the complete gene coding for the α chain, the sequence shown in positions 805 to 867 is the gene coding for the T2A self-cleaving polypeptide, and the sequence shown in positions 868 to 1785 is the complete gene coding for the β chain.

The retroviral vector MSCV-IRES-GFP is a recombinant plasmid obtained by replacing the small DNA fragment between the XhoI and ClaI restriction sites of the MO vector with an IRES nucleotide sequence (Genbank: MG550106.1) and a fluorescent marker protein GFP nucleotide sequence (Genbank: MH777595.1).

The MO vector is described in Tanyu Hu, Krung Phiwpan, Jitao Guo, et al., MicroRNA-142-3p Negatively Regulates Canonical Wnt Signaling Pathway, PLOS ONE, DOI: 10.1371/journal.pone.0158432 (2016).

3. Jurkat cells were cultured to a number of 2×10⁷ or more, and the cells were harvested and washed twice with antibiotic-free 1640 medium. Then the cells were resuspended in antibiotic-free 1640 medium to 5×10⁷ cells/mL after the last wash. The cells were divided into 400 μL aliquots and an aliquot was added to each cuvette (BIO-RAD, catalog number: 165-2088), and 40 μg of the recombinant plasmid MSCV-TCR-GFP was added at the same time, and mixed well. The cuvette was placed in an electroporator (BIO-RAD, Gene Pulser Xcell™), electroporation was carried out at a voltage of 250 V and a capacitance of 950 μF to introduce the recombinant plasmid MSCV-TCR-GFP into Jurkat cells to obtain Jurkat cells expressing TCR.

According to the above method, except for the replacement of the recombinant plasmid MSCV-TCR-GFP with the retroviral vector MSCV-NGFR-GFP, the above steps were repeated to obtain Jurkat cells expressing NGFR.

The retroviral vector MSCV-NGFR-GFP is a recombinant plasmid obtained by replacing the small DNA fragment between the XhoI and EcoRI restriction sites of the MO vector with the NGFR nucleotide sequence (derived from the Addgene NGFR plasmid, Plasmid #27489), and then replacing the small DNA fragment between the EcoRI and ClaI restriction sites with the IRES nucleotide sequence (Genbank: MG550106.1) and a fluorescent marker protein GFP nucleotide sequence (Genbank: MH777595.1).

4. The Jurkat cells expressing TCR or the Jurkat cells expressing NGFR obtained in step 3 were stained with Tetramer (HLA-A11/HBc141-151).

The staining results are shown in FIG. 2. The results show that the TCR had a strong affinity for the HLA-A11-restricted epitope.

II. Construction and Identification of HBc141-151-Specific TCR Transgenic Mice (Hereinafter Referred to as TCR Transgenic Mice)

1. Construction of Recombinant Plasmid phCD2-TCR-α and Recombinant Plasmid p428-TCR-β

(1) The TCR-α gene sequence shown in SEQ ID NO. 1 was inserted into the recognition site of the restriction endonuclease EcoRI of the phCD2 plasmid, and the rest sequence remained unchanged to obtain the recombinant plasmid phCD2-TCR-α.

The phCD2 plasmid is described in Zhumabekov T., Corbella P., Tolaini M. and Kioussis D., Improved Version of a Human CD2 Minigene Based Vector for T Cell-specific Expression in Transgenic Mice, J. of Immunological Methods, September 11;185(1): 133-40 (1995).

(2) The TCR-β gene sequence shown in SEQ ID NO. 3 was inserted into the recognition site of the restriction endonuclease SalI of the p428 plasmid, and the rest of the sequence remained unchanged to obtain the recombinant plasmid p428-TCR-β.

The p428 plasmid is described in Sawada S, Scarborough J D, Killeen N, Littman D R, A Lineage-specific Transcriptional Silencer Regulates CD4 Gene Expression During T Lymphocyte Development, Cell, June 17;77(6):917-29 (1994).

2. Superovulation and Preparation of Pseudopregnant Female Mice

(1) Superovulation

3-week-old C57BL/6 female mice were intraperitoneally injected with PMSG (5 U/mouse); 48 h later, they were intraperitoneally injected with HCG (5 U/mouse); immediately after HCG injection, they were caged with B6D2F1 male mice at a ratio of 1:1 for 18 h to 24 h; vaginal plugs were then examined, and the mice with a vaginal plug were separated for later use.

(2) Preparation of Pseudopregnant Female Mice

In step (1), while C57BL/6 female mice and B6D2F1 male mice were caged together, ICR female mice and ligated ICR male mice were caged at a ratio of 2:1 for 18 h to 24 h; vaginal plugs were then examined, and the mice with a vaginal plug (i.e., pseudopregnant female mice) were separated for later use.

3. Egg Retrieval

The C57BL/6 female mice with vaginal plug in step 2, (1) were sacrificed in a cervical dislocation mode, and the mice were dissected. The upper part of the uterus was clamped with forceps, the fallopian tubes were separated into M2 medium, and the ampulla of the fallopian tubes were cut open under the microscope to allow the eggs to flow into the culture medium. 1 mg/mL hyaluronidase solution was added to the culture medium to remove granulosa cells around the fertilized eggs. The fertilized eggs in better condition were selected and transferred to droplets of M2 medium covered with mineral oil in a plastic dish (diameter, 35 mm) and cultured in a carbon dioxide incubator (37° C., 5% carbon dioxide, 95% air) until the fertilized eggs were suitable for injection.

4. Injection of DNA Solution into Pronucleus of Fertilized Eggs

First, the piston of a 1 mL syringe was cut into two small columns with a length of 0.5 cm, and a coverslip with a width of 0.5 cm was cut with a grinding wheel and sterilized with 75% (v/v) ethanol aqueous solution. Petroleum jelly was used to glue the two cut columns to a glass slide along the length of the glass slide. A 1 mL syringe was used to drop two small drops of M2 solution in the center of the two small columns and drop one drop on the coverslip at the same time. The coverslip was inverted to cover the two small columns and pressed tightly, the syringe needle was inserted into the M2 droplet on the coverslip and the M2 solution was slowly pushed, so that the enclosed chamber was filled with the M2 solution. The completed injection chamber was then placed on the objective table of the micromanipulator, and a group of fertilized eggs (about 100 eggs) was moved into the injection chamber using an embryo transfer tube. The fixation needle and the injection needle were mounted and adjusted to the center of the field of view, and the respective X, Y, and Z axes were adjusted so that the fixation needle, the injection needle and the zygotes were on the same level. The injection needle was pushed through the zona pellucida and into the pronucleus, and the DNA solution (recombinant plasmid phCD2-TCR-α at a concentration of 3-5 ng/μL mixed with recombinant plasmid p428-TCR-β at a concentration of 3-5 ng/μL in a volume ratio of 1:1) was injected into the pronucleus using the continuous pressure of the pressure pump (about 150 hPa) until the pronucleus was slightly enlarged. The injection needle was quickly withdrawn. The fixation needle was adjusted to positive pressure to make the injected zygote fall off, and then adjusted to negative pressure to adsorb another zygote for injection. After the zygotes were injected, they were immediately transferred back to the M16 medium and cultured in an incubator at 37° C. (for about 12 h).

5. Fertilized Egg Transfer

The pseudopregnant female mice were anesthetized, and the ovaries together with the fallopian tubes were surgically removed and fixed with fat forceps. The openings of the fallopian tubes were found under the microscope. The transfer tube was used to absorb the fertilized eggs that have been cultured after microinjection of DNA, the mouth of the transfer tube was inserted into the opening of the fallopian tube and gently blows the fluid in the transfer tube. The ovaries together with the fallopian tubes were put back into the abdominal cavity, and the muscles and skin were sutured.

6. Identification of TCR Transgenic Mice

The genotype identification of the founder mice delivered by the pseudopregnant female mice after the treatment in step 5 was carried out. The specific steps were as follows: tail vein blood was taken from the founder mice, erythrocytes were lysed with ACK erythrocyte lysis buffer, centrifuged, and leukocytes were collected. The obtained leukocytes were stained with anti-mouse CD8a (Biolegend, clone No.: 53-6.7) and anti-mouse TCRVβ10 (BD Bioscience, clone No.: B21.5) antibodies.

According to the above method, except for the replacement of the founder mice with wild-type mice, the above steps were repeated to serve as a control.

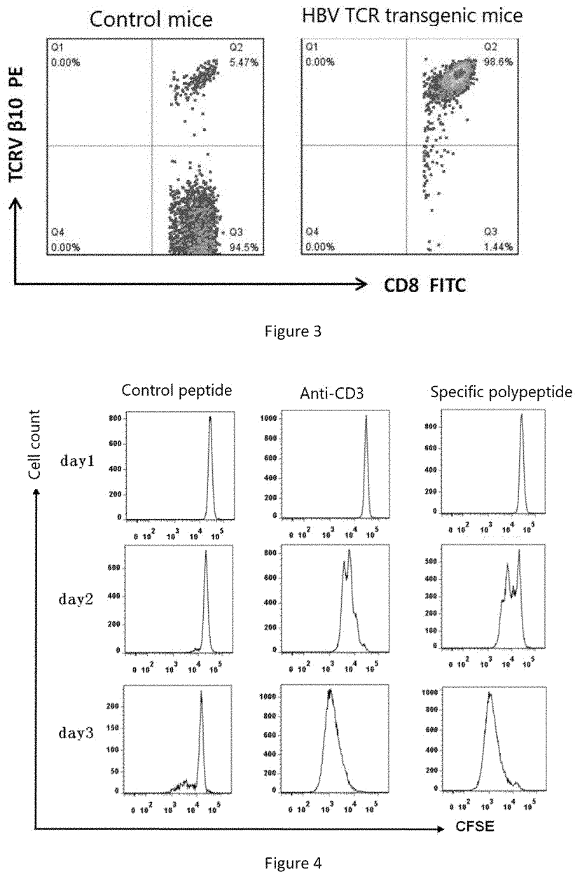

The staining results are shown in FIG. 3. The results show that the high expression of specific TCRVβ in CD8 cells could be detected in TCR transgenic mice in vivo.

IV. CD8+ T Cells of TCR Transgenic Mice (i.e., TCR-T Cells) Exhibited HBV Polypeptide-Dependent Activation and Proliferation Ability

The specific TCR transgene-positive T cells in TCR transgenic mice are TCR-T cells.

1. The cells from the lymph node and spleen of TCR transgenic mice were collected, counted, and diluted to 3×10⁷ cells/mL. 1 μg of anti-CD4 antibody (BioXcell, clone No.: GK1.5) was added to every 3×10⁷ cells and incubated with rotation at 4° C. for 30 min.

2. After the completion of step 1, 2% FBSDMEM medium was added to bring the volume to 15 mL, centrifuged at 4° C. and 2000 rpm for 2 min, and the supernatant was removed to wash off unbound antibodies. Then 1 mL of goat anti-mouse IgG (QIAGEN, 310007), 1 mL of goat anti-rat IgG (QIAGEN, Cat. No.: 310107) and a magnetic bead resuspension (prepared as follows: magnetic beads were washed twice in 1×PBS buffer containing 0.5% (v/v) BSA and 2 mM EDTA, then resuspended in 1×PBS buffer containing 0.5% (v/v) BSA and 2 mM EDTA) were added per 3×10⁷ cells and incubated with rotation at 4° C. for 30 min.

3. After the completion of step 2, the magnet was used to adsorb the magnetic beads to remove CD4+ T cells and B cells. About 90% of the remaining cells in the supernatant were CD8+ T cells, and then the obtained CD8+ cells were labeled with CFSE. The basic process of CFSE labeling was as follows: the cells to be labeled were added with a CFSE diluent or a CFSE stock solution as needed, mixed well, and incubated at 37° C. and 5% CO₂ for 10 min; then 10 volumes of pre-warmed 1640 complete medium were added to stop the labeling and the cells were resuspended in PBS to obtain CFSE-labeled TCR-T cells.

4. The spleen cells of HLA-A11/hTAP-LMP transgenic mice were collected, erythrocytes were lysed, and the cells were counted. Fresh 1640 medium was added to dilute the cells to 1×10⁷ cells/mL, polypeptide HBc141-151 was added to the spleen cells of the HLA-A11/hTAP-LMP transgenic mice to a concentration in the system of 10 μg/mL and cultured at 37° C. and 5% CO₂ for 1 h.

5. After the completion of step 4, the cells were washed twice with 1640 medium and counted.

6. The cells obtained in step 5 and CFSE-labeled TCR-T cells were mixed in a ratio of 1:1 and cultured at 37° C. and 5% C02 for 1 day, 2 days, or 3 days.

7. After the completion of step 6, flow cytometry was performed to analyze the proliferation and activation of TCR-T cells.

Except for the substitution of the polypeptide HBc141-151 in the above method with a control polypeptide, the above steps were repeated to serve as a control. The control polypeptide was NP91 (NP91 is the 91-99 peptide segment of the NP protein of PR8 influenza virus, synthesized by Beijing Xuheyuan Biotechnology Co., Ltd.).

Except for the substitution of the polypeptide HBc141-151 in the above method with an anti-CD3 antibody (clone No. 145-2C11, Bioxcell), the above steps were repeated to serve as a positive control.

The detection results are shown in FIG. 4 (the specific polypeptide is the polypeptide HBc141-151 and the anti-CD3 is the anti-CD3 antibody). The results show that HBc141-151 effectively activates TCR-T cells and effectively stimulates the proliferation of TCR-T cells. It can be seen that TCR-T cells had HBV polypeptide-dependent activation and proliferation ability in vitro. When TCR-T cells were reinfused into HBV-positive animals or patients, they could effectively activate and expand the reinfused TCR-T cells in vivo.

V. TCR-T Cells have the Ability to Kill Target Cells In Vitro

In order to verify the ability of TCR to kill target cells in vitro, an in vitro killing experiment was performed. The specific steps were as follows:

1. Activation of TCR-T Cells

(1) The cells from the lymph node and spleen of TCR transgenic mice were collected and treated with magnetic beads capable of binding to CD4+ T cells and B cells, so as to remove CD4+ T cells and B cells and enrich CD8+ T cells, and then the cells were counted.

(2) The spleen cells of HLA-A11/hTAP-LMP transgenic mice were collected, erythrocytes were lysed, and the cells were counted. Fresh 1640 medium was added to dilute the cells to 1×10⁷ cells/mL, and polypeptide HBc141-151 was added to the spleen cells of the HLA-A11/hTAP-LMP transgenic mice to a concentration of 10 μg/mL in the system and cultured at 37° C. and 5% CO₂ for 1 h.

(3) After the completion of step (2), the cells were washed twice with 1640 medium and counted.

(4) The cells obtained in step (3) and the CD8+ T cells of TCR transgenic mice were mixed in equal quantities, added with 400 U/mL IL-2, and cultured at 37° C. and 5% CO₂ for 5 days to obtain in vitro activated TCR-T cells.

2. The spleen cells of the HLA-A11/hTAP-LMP transgenic mice were collected, erythrocytes were lysed, and the cells were counted. Fresh 1640 medium was added to dilute the cells to 1×10⁷ cells/mL, and polypeptide HBc141-151 was added to the spleen cells of the HLA-A11/hTAP-LMP transgenic mice to a concentration of 10 μg/mL in the system and cultured at 37° C. and 5% CO₂ for 1 h. Then, the cells were washed twice with 1640 medium and counted to obtain the target cells loaded with polypeptide HBc141-151.

3. The target cells loaded with polypeptide HBc141-151 obtained in step 2 were added to a 96-well plate, 1×10⁴ cells/well; then in vitro activated TCR-T cells were added at a ratio of 10:1, 5:1 or 1:1 and cultured at 37° C. and 5% CO₂ for 5 h.

4. The 96-well plate was centrifuged at 250 g for 4 min; then 50 μL of supernatant was transferred to a new 96-well plate, 50 μL of substrate was added to each well, and the 96-well plate was incubated at room temperature for 30 min in the dark; then 50 μL of stop solution was added to each well, and the absorbance at 490 nm wavelength, i.e., experimental group value, was measured using an enzyme-labeling instrument.

The substrate, stop solution, and cell lysis buffer were all obtained from a CytoTox 96® Non-Radioactive Cytotoxicity Assay Kit (Promega, Cat. No. G1780).

5. Background Value Detection of Killing Experiment

(1) According to the above steps 1-4, except that step 3 was replaced with step 3A), the above steps were repeated to obtain the T cell self-release value. Step 3A): the same number of in vitro activated TCR-T cells as the experimental group were added to a 96-well plate and cultured at 37° C. and 5% CO₂ for 5 hours.

(2) According to the above steps 1-4, except that step 3 was replaced with step 3B), the above steps were repeated to obtain the target cell self-release value.

Step 3B): the same number of target cells loaded with polypeptide HBc141-151 as the experimental group were added to a 96-well plate and cultured at 37° C. and 5% CO₂ for 5 hours.

(3) According to the above steps 1-4, except that step 3 was replaced with step 3C), the above steps were repeated to obtain the target cell maximum release value.

Step 3C): the same number of target cells loaded with polypeptide HBc141-151 as the experimental group and 10 μL of cell lysis buffer were added to a 96-well plate and cultured at 37° C. and 5% CO₂ for 5 hours.

6. Calculation of Killing Activity

Killing activity=(experimental group value−T cell self-release value−target cell self-release value)/(target cell maximum release value−target cell self-release value).

Except that the polypeptide HBc141-151 in the above method was replaced with NP91, the above steps were repeated to serve as a control.

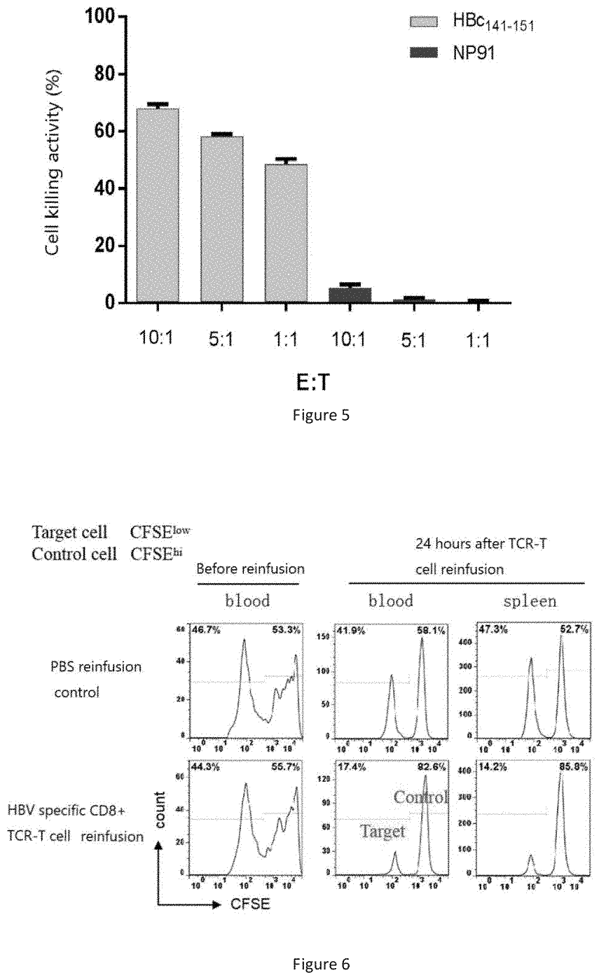

The detection results are shown in FIG. 5 (E:T represents the ratio of TCR-T cells to target cells). The results show that TCR-T cells were able to effectively kill target cells in vitro.

VI. TCR-T Cells are Effective in Killing Target Cells In Vivo

In order to verify the ability of TCR to specifically kill target cells in vivo, an in vivo killing experiment was performed. The HLA-A11/hTAP-LMP cells loaded with the polypeptide HBc141-151 were labeled with a lower concentration of CFSE (CFSElow), and the HLA-A11/hTAP-LMP cells unloaded with polypeptide (used as control) were labeled with a higher concentration of CFSE (CFSEhigh). The two cells were mixed in equal amounts and then reinfused into C57/B6J mice, and then preactivated TCR-T cells were reinfused. Flow cytometer was used to detect whether the CFSElow-labeled cell population in C57/B6J mice would be killed by TCR-T cells. The specific steps were as follows:

1. Activation of TCR-T Cells

(1) The cells from the lymph node and spleen of TCR transgenic mice were collected and treated with magnetic beads capable of binding to CD4+ T cells and B cells, so as to remove CD4+ T cells and B cells and enrich CD8+ T cells, and then the cells were counted.

(2) The spleen cells of HLA-A11/hTAP-LMP transgenic mice were collected, erythrocytes were lysed, and the cells were counted. Fresh 1640 medium was added to dilute the cells to 1×10⁷ cells/mL, and polypeptide HBc141-151 was added to the spleen cells of the HLA-A11/hTAP-LMP transgenic mice to a concentration of 10 μg/mL in the system and cultured at 37° C. and 5% CO₂ for 1 h.

(3) After the completion of step (2), the cells were washed twice with 1640 medium and counted.

(4) The cells obtained in step (3) and the CD8+ T cells of TCR transgenic mice were mixed in equal quantities, added with 400 U/mL IL-2, and cultured at 37° C. and 5% CO₂ for 5 days to obtain in vitro activated TCR-T cells.

2. Preparation of Target Cells

(1) The spleen cells of HLA-A11/hTAP-LMP transgenic mice were collected, erythrocytes were lysed, and the cells were counted. Fresh 1640 medium was added to dilute the cells to 1×10⁷ cells/mL, and polypeptide HBc141-151 was added to the spleen cells of the HLA-A11/hTAP-LMP transgenic mice to a concentration of 10 μg/mL in the system and cultured at 37° C. and 5% CO₂ for 1 h. Then, the cells were washed twice with 1640 medium and counted.

(2) After the completion of step (1), the cells were resuspended in PBS to obtain a spleen cell loaded with polypeptide HBc141-151 resuspension with a concentration of 5×10⁷ cells/mL.

(3) After the completion of step (2), the spleen cells loaded with polypeptide HBc141-151 were labeled with a lower concentration of CFSE (CFSElow) (for each mL of spleen cells loaded with polypeptide HBc141-151, 1 μL of 0.5 mM CFSE diluent should be added; when CFSE labeling was performed, the concentration of CFSE was 0.5 μM) to obtain spleen cells loaded with polypeptide HBc141-151 and labeled with CFSElow.

(4) The spleen cells of HLA-A11/hTAP-LMP transgenic mice were collected, erythrocytes were lysed, and the cells were counted; and then the cells were cultured at 37° C. and 5% CO₂ for 1 h (in 1640 medium).

(5) After the completion of step (4), the cells were washed twice with 1640 medium and counted.

(6) After the completion of step (5), the cells were resuspended in PBS buffer to obtain a cell resuspension with a concentration of 5×10⁷ cells/mL.

(7) After the completion of step (6), the spleen cells unloaded with polypeptide were labeled with a higher concentration of CFSE (CFSEhigh) (for each mL of spleen cells unloaded with polypeptide, 1 μL of 5 mM CFSE stock solution should be added; when CFSE labeling was performed, the concentration of CFSE was 5 μM) to obtain spleen cells labeled with CFSEhigh.

3. TCR-T Cells are Effective in Killing Target Cells In Vivo

(1) The spleen cells loaded with polypeptide HBc141-151 and labeled with CFSElow and the spleen cells labeled with CFSEhigh were mixed in equal quantities to obtain mixed cells.

(2) Each C57/B6J mouse was reinfused (via the tail vein) with 2×10⁷ mixed cells.

(3) 2 hours after the completion of step (2), each C57/B6J mouse was reinfused (via the tail vein) with 1×10⁷ in vitro activated TCR-T cells obtained in step 1.

(4) 24 hours after the completion of step (3), the mice were sacrificed, peripheral blood and spleen cells were harvested, erythrocytes were lysed and centrifuged to obtain leukocytes, and a single cell suspension was prepared. The ratio of CFSElow to CFSEhigh in peripheral blood and spleen was detected by flow cytometry. The killing function of TCR-T cells was quantitatively analyzed by the ratio of CFSElow to CFSEhigh.

According to the above steps (1)-(4), except that step (3) was replaced with step K, the above steps were repeated to serve as a PBS reinfusion control. Step K: 2 hours after the completion of step (2), each C57/B6J mouse was reinfused with the same volume of PBS buffer as the in vitro activated TCR-T cells in step (3).

The test results are shown in FIG. 6. Compared with the PBS reinfusion control group, whether in peripheral blood or spleen, the CFSElow-labeled cell population was significantly decreased compared with the CFSEhigh cell population. The results show that TCR-T cells were able to effectively kill target cells in vivo.

VII. TCR-T Cells can Effectively Clear Chronic HBV Infection In Vivo

1. Establishment of a Mouse Model of Chronic HBV Infection

The method of establishing a mouse model of chronic HBV infection is described in Dong Xiaoyan, Yu Chijie, Wang Gang, et al., The in vivo Transduction Method of Highly Hepatotropic Type 8 Recombinant Adeno-associated Virus for Preparing a Mouse Model of Persistent Hepatitis B Virus Infection, Virology, 26(6): 425-431 (2010). The specific steps were as follows:

(1) rAAV/HBV1.3 virus (obtained from Beijing FivePlus Molecular Medicine Institute Co. Ltd.) was injected into the tail vein of HLA-A11/hTAP-LMP transgenic mice at a dose of 5×10⁹ vg/mouse.

After the completion of step (1), the change curve of the virus gradient (HBsAg, HBeAg or HBV DNA) under natural conditions was observed, and the administration time of TCR-T cells was predicted according to the change curve.

(2) One month after the completion of step (1), a mouse model of chronic HBV infection was obtained. The expression of HBsAg and HBeAg in the mouse model of chronic HBV infection was regulated by elements such as the promoter of the HBV DNA itself. Continuously expressed HBsAg and HBeAg could be observed in the liver and peripheral blood for 10 weeks.

Except for the replacement of the HLA-A11/hTAP-LMP transgenic mice with C57BL/6 mice, the above steps were repeated to obtain chronic HBV-infected C57BL/6 mice as a negative control.

2. Treatment of Chronic HBV Infection in HLA-A11/hTAP-LMP Mice with TCR-T Cells

(1) The cells from the lymph node and spleen of TCR transgenic mice were collected and treated with magnetic beads capable of binding to CD4+ T cells and B cells, so as to remove CD4+ T cells and B cells and enrich CD8+ T cells, and then the cells were counted.

(2) The spleen cells of HLA-A11/hTAP-LMP transgenic mice were collected, erythrocytes were lysed, and the cells were counted. Fresh 1640 medium was added to dilute the cells to 1×10⁷ cells/mL, and polypeptide HBc141-151 was added to the spleen cells of the HLA-A11/hTAP-LMP transgenic mice to a concentration of 10 μg/mL in the system and cultured at 37° C. and 5% CO₂ for 1 h.

(3) After the completion of step (2), the cells were washed twice with 1640 medium and counted. Then, the cells and the CD8+ T cells of TCR transgenic mice were mixed in equal quantities, added with 400 U/mL TL-2, and cultured at 37° C. and 5% CO₂ for 5 days to obtain in vitro activated TCR-T cells.

(4) 1×10⁷ in vitro activated TCR-T cells were reinfused (via the tail vein) into each HLA-A11/hTAP-LMP transgenic mouse model of chronic HBV infection.

(5) The day when step (4) was completed was recorded as day 0, and the OD450 nm value of HBsAg (HBsAg detection kit obtained from Shanghai Kehua) and ALT concentration (ALT/GPT kit, automatic biochemical analyzer MEDSOUL AMS-18) in serum were measured every two days (for a total of 8 times), so as to determine the infection status of HBV virus in vivo.

(6) On day 7 after the completion of step (4), 1×10⁷ in vitro activated TCR-T cells were reinfused into each HLA-A11/hTAP-LMP transgenic mouse model of chronic HBV infection for the second time.

(7) Two days after the completion of step (6), the OD450 nm value of HBsAg and ALT concentration in serum were detected.

(8) Except that the HLA-A11/hTAP-LMP transgenic mouse model of chronic HBV infection in steps (4)-(7) was replaced with the C57BL/6 mouse model of chronic HBV infection, the above steps were repeated to serve as a control.

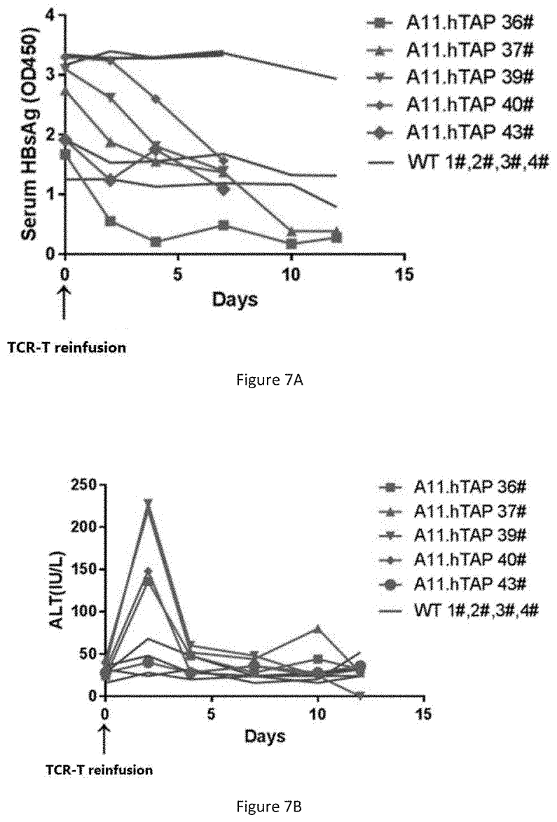

The detection results of the OD450 nm value of HBsAg in serum are shown in FIGS. 7A and 7B, panel FIG. 7A (WT 1 #, 2 #, 3 # and 4 # are all C57BL/6 mouse models of chronic HBV infection, A11.hTAP 36 #, A11.hTAP 37 #, A11.hTAP 39 #, A11.hTAP 40 # and A11.hTAP 43 # are all HLA-A11/hTAP-LMP transgenic mouse models of chronic HBV infection). The detection results of ALT concentration in serum are shown in FIGS. 7A and 7B, panel FIG. 7B (WT 1 #, 2 #, 3 # and 4 # are all C57BL/6 mouse models of chronic HBV infection, A11.hTAP 36 #, A11.hTAP 37 #, A11.hTAP 39 #, A11.hTAP 40 # and A11.hTAP 43 # are all HLA-A11/hTAP-LMP transgenic mouse models of chronic HBV infection). The results show that TCR-T cells could effectively clear the HBV infection in HLA-A11/hTAP-LMP transgenic mouse model of chronic HBV infection.

VIII. Human TCR-T Cells have the Ability to Kill Target Cells In Vitro

In order to verify the ability of human TCR-T cells to kill target cells in vitro, an in vitro killing experiment was performed. The specific steps were as follows:

1. Packaging and Concentration of Lentivirus

(1) The small DNA fragment between the EcoRI and BamHI restriction sites of the lentiviral packaging vector pCDH-MSCV-MCS-IRES-GFP (SystemBiosciences, ID: CD731B-1) was replaced with a TCR DNA fragment (SEQ ID NO. 5) to obtain the pCDH-MSCV-TCR-GFP plasmid.

(2) 293T cells were pipetted up and down and resuspended to obtain a single cell suspension. The cells were counted and then suspended in DMEM medium containing 10% (v/v) FBS to obtain a cell suspension with a concentration of 5×10⁵ cells/mL. 10 mL of the cell suspension was spread on a 10 cm-diameter culture dish and cultured overnight.

(3) After the completion of step (2), transfection was performed when the confluence of 293T cells was 75%, and the medium was replaced with DMEM medium 30 min before transfection.

(4) Preparation of Transfection Premix

To 500 μL of DMEM medium, 12 μg of pCDH-MSCV-TCR-GFP plasmid, 9 μg of psPAX2 and 6 μg of pMG2.D were added, and vortexed to mix well to obtain a plasmid mixture.

psPAX2 and pMG2.D, were obtained from Beijing Tiandz Gene Technology Co., Ltd.

27 μg of PEI was added to 500 μL of DMEM medium, vortexed and mixed, and allowed to stand for 5 min to obtain a PEI mixture.

(5) After the completion of step (4), 500 μL of the plasmid mixture was added with 500 μL of the PEI mixture, vortexed to mix well, and incubated at room temperature for 20 min; then the incubated mixture was gently added to the 293T cells along the side wall of the culture dish, and the culture dish was gently shaken and incubated in a 37° C. incubator. After 6 h to 8 h, the medium was replaced with 10 mL of DMEM medium containing 10% (v/v) FBS.

(6) 48 h after the completion of step (5), the first virus supernatant was collected, supplemented with fresh DMEM medium, and the virus supernatant was stored at 4° C.

(7) 72 h after the completion of step (5), the second virus supernatant was collected. The first virus supernatant and the second virus supernatant were pooled, centrifuged at 800 g for 5 min at room temperature, and the supernatant was collected.

(8) After the completion of step (7), the supernatant was filtered using a 0.45 m PES filter to remove cell debris, and the virus supernatant was collected.

(9) After the completion of step (8), the virus supernatant was transferred to an ultracentrifuge tube. The tube was then centrifuged at 70000 g at 4° C. for 120 min, the supernatant was carefully discarded, and the precipitate (containing white virus particles) was collected.

(10) After the completion of step (9), 100 concentrated volumes of 1640 medium were added to the precipitate to resuspend the virus particles, and the virus particles were dissolved overnight at 4° C. to obtain concentrated lentiviruses. The concentrated lentiviruses were sub-packaged and stored in a −80° C. ultra-low temperature freezer for later use.

2. Isolation of Human Peripheral Lymphocytes

(1) 5 mL of human peripheral venous blood was drawn.

(2) After the completion of step (1), 5 mL of peripheral blood and 5 mL of PBS buffer were added to a 50 mL centrifuge tube and mixed thoroughly.

(3) After the completion of step (2), 5 mL of human peripheral blood lymphocyte separation solution (Tianjin Haoyang Biological Manufacture Co., Ltd., Cat. No.: LTS 1077) was added into the centrifuge tube, and a disposable sterile dropper was used to take the diluted peripheral blood and carefully superimpose it on top of the separation solution surface along the tube wall. Particular attention was paid to keeping the interface clear.

(4) After the completion of step (3), the centrifuge tube was placed in the centrifuge, the speed up and speed down were adjusted to the minimum, and the centrifuge tube was centrifuged at 800 g for 20 min.

(5) After the completion of step (4), the liquid in the tube was divided into four layers after the centrifugation, the first layer was plasma and PBS, the second layer was the annular milky white lymphocyte layer, the third layer was the transparent separation liquid layer, and the fourth layer was the erythrocyte and granulocyte layer. The second annular milky white lymphocyte layer was carefully transferred to a 50 mL sterile centrifuge tube with a pipette, and 40 mL of PBS buffer was added to the centrifuge tube. The cells were mixed and centrifuged at 800 g for 5 min and the supernatant was discarded. The cells were then resuspended in 1640 medium and counted for later use.

3. Activation of Human Peripheral T Lymphocytes

(1) In a 24-well plate, 500 μL of anti-CD3 antibody diluent and 500 μL of anti-CD28 antibody diluent were added to each well, and coated overnight at 4° C.

Anti-CD3 antibody diluent: anti-CD3 antibody (BioXcell, clone No.: OKT3) was diluted with PBS buffer to a concentration of 3 μg/mL.

Anti-CD28 antibody diluent: anti-CD28 antibody (BioXcell, clone No.: CD28.2) was diluted with PBS buffer to a concentration of 1 μg/mL.

(2) After the completion of step (1), the liquid in the 24-well plate was removed and the 24-well plate was wash once with PBS buffer.

(3) After the completion of step (2), 500 μL of human peripheral T lymphocyte diluent was added to the 24-well plate and cultured in a 37° C. incubator for 48 h; then the 24-well plate was centrifuged at 400 g for 5 min, the precipitate was collected and resuspended in 1640 medium to obtain activated human peripheral T lymphocytes. The activated human peripheral T lymphocytes were used for infection with lentiviruses.

Human peripheral T lymphocyte diluent: the human peripheral T lymphocytes obtained in step 2 were diluted with 1640 medium to 4×10⁶ cells/mL.

4. Lentiviral Infection of Human Peripheral T Lymphocytes

(1) In a 24-well plate, 5×10⁵ activated human peripheral T lymphocytes and 200 L of concentrated lentiviruses were added to each well, and then supplemented with 1640 medium to a final volume of 500 μL; finally, polybrene and IL2 were added, such that the concentrations of polybrene and IL2 in the system were 8 μg/mL and 40 U/mL, respectively.

(2) After the completion of step (1), the 24-well plate was centrifuged at 600 g at 32° C. for 90 min and then placed in a 37° C. incubator for 24 hours of infection.

(3) After the completion of step (2), 350 μL of medium in the infected well was carefully removed from the 24-well plate, 1640 medium was added to bring the volume to 2 mL and mixed by pipetting. The 24-well plate was then placed in a 37° C. incubator for 48 hours.

(4) After the completion of step (3), an appropriate amount of infected T cells was collected for flow cytometry to detect the infection efficiency; then the infected cells were stained with anti-mouse CD8a antibody (Biolegend, clone No.: 53-6.7) and anti-mouse TCRVβ10 antibody (BD Bioscience, clone No.: B21.5). After 30 minutes of staining at 4° C., the cells were detected with flow cytometry. The infection efficiency (TCRVβ10 positive rate) was greater than 15%, and the positive cells were human TCR-T cells that were successfully introduced with TCR for subsequent killing experiment.

5. Human TCR-T have the Ability to Kill Target Cells In Vitro

The substrate, stop solution and cell lysis buffer are all components of the CytoTox 96® Non-Radioactive Cytotoxicity Assay Kit (Promega, Cat. No.: G1780).

(1) 5 mL of peripheral venous blood was drawn from an HLA-A11 positive healthy person, and then lymphocytes, i.e., HLA-A11+ PBMC cells, were isolated according to the method in step 2.

(2) After the completion of step (1), the HLA-A11+ PBMC cells were diluted with 1640 medium to a concentration of 1×10⁷ cells/mL, and then the polypeptide HBc141-151 was added to a concentration of 10 μg/mL in the system; the cells were cultured at 37° C. and 5% CO₂ for 1 h, then washed twice with 1640 medium, and counted to obtain target cells loaded with polypeptide HBc141-151.

(3) The target cells loaded with polypeptide HBc141-151 obtained in step (2) were added into a 96-well plate, 2×10⁴ cells/well; then human TCR-T cells at a were added to the target cells at a ratio of 2:1 or 1:1, cultured at 37° C. and 5% CO₂ for 5 h.

(4) The 96-well plate was centrifuged at 250 g for 4 min; then 50 μL of supernatant was transferred to a new 96-well plate, 50 μL of substrate was added to each well, and incubated at room temperature for 30 min in the dark; then 50 μL of stop solution was added to each well, and the absorbance at 490 nm wavelength, i.e., experimental group value, was measured using an enzyme-labeling instrument.

(5) Background Value Detection of Killing Experiment

1. According to the above steps (1)-(4), except that step (3) was replaced with step 3A), the above steps were repeated to obtain the T cell self-release value.

Step 3A): the same number of human TCR-T cells as the experimental group were added to a 96-well plate and cultured at 37° C. and 5% CO₂ for 5 hours.

2. According to the above steps (1)-(4), except that step (3) was replaced with step 3B), the above steps were repeated to obtain the target cell self-release value.

Step 3B): the same number of target cells loaded with polypeptide HBc141-151 as the experimental group were added to a 96-well plate and cultured at 37° C. and 5% CO₂ for 5 hours.

3. According to the above steps (1)-(4), except that step (3) was replaced with step 3C), the above steps were repeated to obtain the target cell maximum release value.

Step 3C): the same number of target cells loaded with polypeptide HBc141-151 as the experimental group and 10 μL of cell lysis buffer were added to a 96-well plate and cultured at 37° C. and 5% CO₂ for 5 hours.

6. Calculation of Killing Activity

Killing activity=(experimental group value−T cell self-release value−target cell self-release value)/(target cell maximum release value−target cell self-release value)

Except that the polypeptide HBc141-151 in the above method was replaced with NP91, the above steps were repeated to serve as a control.

Except that the human TCR-T cells in the above method were replaced with untransfected human T cells, the above steps were repeated to serve as a control.

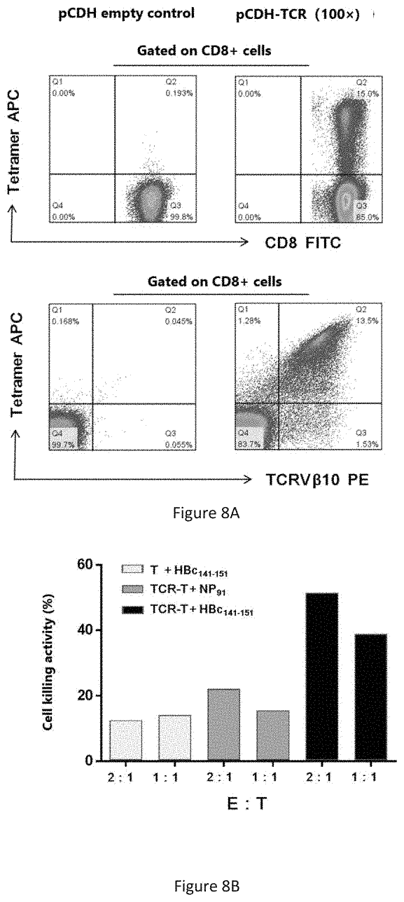

The test results are shown in FIGS. 8A and 8B (panel FIG. 8A shows the acquisition of human TCR-T cells; panel FIG. 8B shows the in vitro killing experiment of target cells by human TCR-T cells, wherein E:T represents the ratio of human TCR-T cells to target cells, T+HBc141-151 represents human T cells killing target cells incubated with HBc141-151 polypeptide, TCR-T+NP91 represents human TCR-T cells killing control cells incubated with NP91 polypeptide, and TCR-T+HBc141-151 represents human TCR-T cells killing target cells incubated with HBc141-151 polypeptide). The results show that human TCR-T cells could effectively kill target cells in vitro.

In conclusion, the inventors of the present invention have isolated and identified a pair of HBV-specific TCR sequences, successfully constructed transgenic mice that express this pair of TCRs, and verified in vitro that TCR transgene-positive CD8 cells (i.e., TCR-T cells) have HBV polypeptide epitope-dependent activation and proliferation ability in vitro; used animal in vivo and in vitro killing target cell experiments to demonstrate that this pair of TCRs has good target cell killing activity; conducted an in vitro experiment that demonstrated that human TCR-T cells also have the ability to kill HLA-A11+ target cells; and in addition, conducted animal experiments suggesting that this pair of TCR sequences may be useful in methods that can clear HBV-infected cells effectively.

### INDUSTRIAL APPLICATION

The present invention relates to the isolation and identification of a pair of HBV-specific TCR sequences. These isolated TCR sequences were, in turn, used in constructing transgenic mice and verifying, using in vitro experiments, that TCR transgene-positive CD8 cells (i.e., TCR-T cells) exhibit HBV polypeptide epitope-dependent activation and proliferation ability. The utility of the TCR-T cells has been confirmed using both in vivo and in vitro target cell killing experiments that demonstrated that this pair of isolated TCRs exhibit an ability to kill target cells (spleen cells or PBMC cells loaded with polypeptide HBc141-151 in HLA-A11 transgenic mice). The utility of the TCR-T cells was also verified with in vitro experiments using human TCR-T cells that were able to kill target cells (specifically HLA-A11-restricted human PBMC cells loaded with polypeptide HBc141-151). In addition, other animal experiments suggest that this pair of TCR sequences can be utilized in developing more effective methods for clearing HBV-infected cells from patients and reducing the risk of subsequent cancers.

1-17. (canceled)

18. AT cell receptor that recognizes HLA-A11-restricted HBc₁₄₁₋₁₅₁ epitope peptide, which comprises an α chain and a β chain;
wherein the α chain comprises three complementarity determining regions with amino acid sequences shown in positions 48 to 53, positions 71 to 77 and positions 112 to 121 of SEQ ID NO. 2, respectively; or variants of these sequences with up to 3, 2, or 1 amino acid changes;
wherein the β chain comprises three complementarity determining regions with amino acid sequences shown in positions 46 to 50, positions 68 to 73 and positions 111 to 122 of SEQ ID NO. 4, respectively; or variants of these sequences with up to 3, 2, or 1 amino acid changes.

19. The T cell receptor according to claim 18, wherein the amino acid sequence of the variable region of the α chain is shown in positions 22 to 112 of SEQ ID No. 2; or its variant with up to 3, 2, or 1 amino acid changes;
the amino acid sequence of the variable region of the 3 chain is shown in positions 20 to 113 of SEQ ID No. 4; or its variant with up to 3, 2, or 1 amino acid changes.

20. The T cell receptor according to claim 18, wherein the amino acid sequence of the constant region of the α chain is shown in positions 133 to 268 of SEQ ID No. 2;
the amino acid sequence of the constant region of the β chain is shown in positions 133 to 305 of SEQ ID NO. 4.

21. The T cell receptor according to claim 18, wherein the amino acid sequence of the α chain is shown in SEQ ID NO. 2;
the amino acid sequence of the β chain is shown in SEQ ID NO. 4.

22. Biomaterials as described in any of the following:
(A). A nucleic acid molecule coding for the T cell receptor according to claim 18.
(B). An expression cassette, a vector or a cell containing the nucleic acid molecule coding for the T cell receptor according to claim 18.
(C). A T cell having the T cell receptor according to claim 18.
(D). A pharmaceutical composition comprising a vector or a cell containing a nucleic acid molecule coding for the T cell receptor according to claim 18 or comprising T cell having the T cell receptor according to claim 18.

23. The biomaterials according to claim 22, wherein the nucleic acid molecule coding for the T cell receptor comprises a nucleic acid molecule coding for the α chain of the T cell receptor and a nucleic acid molecule coding for the β chain of the T cell receptor;
the nucleotide sequences coding for the three complementarity determining regions in the α chain of the T cell receptor are shown in positions 142 to 159, positions 211 to 231 and positions 334 to 363 of SEQ ID No. 1, respectively; or sequences having at least 99%, 95%, 90%, 85% or 80% identity thereto and encoding the same amino acid residues;
the nucleotide sequences coding for the three complementarity determining regions in the 3 chain of the T cell receptor are shown in positions 136 to 150, positions 202 to 219 and positions 331 to 366 of SEQ ID No. 3, respectively; or sequences having at least 99%, 95%, 90%, 85% or 80% identity thereto and encoding the same amino acid residues.

24. The biomaterials according to claim 22, wherein the nucleotide sequence coding for the variable region of the α chain is shown in positions 64 to 336 of SEQ ID No. 1; or a sequence having at least 99%, 95%, 90%, 85% or 80% identity thereto and encoding the same amino acid residues;
the nucleotide sequence coding for the variable region of the β chain is shown in positions 58 to 339 of SEQ ID No. 3; or a sequence having at least 99%, 95%, 90%, 85% or 80% identity thereto and encoding the same amino acid residues.

25. The biomaterials according to claim 22, wherein the nucleotide sequence coding for the constant region of the α chain is shown in positions 397 to 807 of SEQ ID No. 1;
the nucleotide sequence coding for the constant region of the β chain is shown in positions 397 to 918 of SEQ ID No. 3.

26. The biomaterials according to claim 22, wherein the nucleotide sequence of the nucleic acid molecule coding for the α chain is shown in SEQ ID No. 1;
the nucleotide sequence of the nucleic acid molecule coding for the β chain is shown in SEQ ID No. 3.

27. The biomaterials according to claim 22, wherein the vector is a retroviral vector or a lentiviral vector;
the retroviral vector is a recombinant plasmid obtained by inserting the nucleic acid molecule coding for the α chain of the T cell receptor and the nucleic acid molecule coding for the β chain of the T cell receptor between the multiple cloning sites of the retroviral vector MSCV-IRES-GFP;
the lentiviral vector is a recombinant plasmid obtained by inserting the nucleic acid molecule coding for the α chain of the T cell receptor and the nucleic acid molecule coding for the β chain of the T cell receptor between the multiple cloning sites of the lentiviral packaging vector pCDH-MSCV-MCS-IRES-GFP.

28. The biomaterials according to claim 22, wherein the cell is a T cell or a Jurkat cell.

29. A method for preventing and/or treating diseases caused by HBV infection, comprising the step of using the T cell receptor according to claim 18, or, the biomaterials according to claim 18, to prevent and/or treat diseases caused by HBV infection.

30. The method according to claim 29, wherein the disease caused by HBV infection is chronic hepatitis B or hepatocellular carcinoma.


SEQUENCE LISTING (93 sequences)

SEQ ID NO: 1 - DNA, 807 bp, Artificial Sequence
  Synthesized
atgaagacagtgactggacctttgttcctgtgcttctggctgcagctgaacggtgtgagcagaggcgagcaggtggagcagcgccctcctcacctgagtgtccgggagggagacagtgccgttatcatctgcacctacacagaccctaacagttattacttcttctggtacaagcaagagccgggggcaggtcttcagttgcttatgaaggttttctcaagtacggaaataaacgaaggacaaggattcactgtcctactgaacaagaaagacaaacaactctctctgaacctcacagctgcccatcctggggactcagccgtgtacttctgcgcagtttactcgggatacaacaaactcacttttggaaagggcacggtgcttctagtctctccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatggaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtcctga

SEQ ID NO: 2 - protein, 268 aa, Artificial Sequence
  Synthesized
MKTVTGPLFLCFWLQLNGVSRGEQVEQRPPHLSVREGDSAVIICTYTDPNSYYFFWYKQEPGAGLQLLMKVFSSTEINEGQGFTVLLNKKDKQLSLNLTAAHPGDSAVYFCAVYSGYNKLTFGKGTVLLVSPDIQNPEPAVYQLKDPRSQDSTLCLFTDFDSQINVPKTMESGTFITDKTVLDMEAMDSKSNGAIAWSNQTSFTCQDIFKETNATYPSSDVPCDATLTEKSFETDMNLNFQNLSVMGLRILLLKVAGFNLLMTLRLWS

SEQ ID NO: 3 - DNA, 918 bp, Artificial Sequence
  Synthesized
atgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctatgtgggcgcggacacagaagtcttctttggtaaaggaaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggctatggtcaagaaaaaaaattcctga

SEQ ID NO: 4 - protein, 305 aa, Artificial sequence
  Synthesized
MGCRLLSCVAFCLLGIGPLETAVFQTPNYHVTQVGNEVSFNCKQTLGHDTMYWYKQDSKKLLKIMFSYNNKQLIVNETVPRRFSPQSSDKAHLNLRIKSVEPEDSAVYLCASSYVGADTEVFFGKGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 5 - DNA, 1785 bp, Artificial Sequence
  Synthesized
atgaagacagtgactggacctttgttcctgtgcttctggctgcagctgaacggtgtgagcagaggcgagcaggtggagcagcgccctcctcacctgagtgtccgggagggagacagtgccgttatcatctgcacctacacagaccctaacagttattacttcttctggtacaagcaagagccgggggcaggtcttcagttgcttatgaaggttttctcaagtacggaaataaacgaaggacaaggattcactgtcctactgaacaagaaagacaaacaactctctctgaacctcacagctgcccatcctggggactcagccgtgtacttctgcgcagtttactcgggatacaacaaactcacttttggaaagggcacggtgcttctagtctctccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatggaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccggctccggagagggcagaggaagtctgctaacatgcggtgacgtcgaggagaatcctggcccaatgggctgtaggctcctaagctgtgtggccttctgcctcttgggaataggccctttggagacggctgttttccagactccaaactatcatgtcacacaggtgggaaatgaagtgtctttcaattgtaagcaaactctgggccacgatactatgtattggtacaagcaagactctaagaaattgctgaagattatgtttagctacaataataagcaactcattgtaaacgaaacagttccaaggcgcttctcacctcagtcttcagataaagctcatttgaatcttcgaatcaagtctgtagagccggaggactctgctgtgtatctctgtgccagcagctatgtgggcgcggacacagaagtcttctttggtaaaggaaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggctatggtcaagaaaaaaaattcctga

SEQ ID NO: 6 - DNA, 19 bp, Artificial Sequence
  Synthesized
ggttatcctggtaccagca

SEQ ID NO: 7 - DNA, 20 bp, Artificial Sequence
  Synthesized
catctactggtaccgacagg

SEQ ID NO: 8 - DNA, 15 bp, Artificial Sequence
  Synthesized
ggcgagcaggtggag

SEQ ID NO: 9 - DNA, 21 bp, Artificial Sequence
  Synthesized
tctgstctgagatgcaatttt

SEQ ID NO: 10 - DNA, 26 bp, Artificial Sequence
  Synthesized
ggctacttcccttggtataagcaaga

SEQ ID NO: 11 - DNA, 20 bp, Artificial Sequence
  Synthesized
cagatgcaaggtcaagtgac

SEQ ID NO: 12 - DNA, 18 bp, Artificial Sequence
  Synthesized
aaggtccacagctccttc

SEQ ID NO: 13 - DNA, 21 bp, Artificial Sequence
  Synthesized
gttctggtatgtgcagtatcc

SEQ ID NO: 14 - DNA, 20 bp, Artificial Sequence
  Synthesized
agattccgtgactcaaacag

SEQ ID NO: 15 - DNA, 25 bp, Artificial Sequence
  Synthesized
agaaggtrcagcagagcccagaatc

SEQ ID NO: 16 - DNA, 17 bp, Artificial Sequence
  Synthesized
gagcrtccasgagggtg

SEQ ID NO: 17 - DNA, 18 bp, Artificial Sequence
  Synthesized
ccagtggttcaaggagtg

SEQ ID NO: 18 - DNA, 19 bp, Artificial Sequence
  Synthesized
agagaaggtcgagcaacac

SEQ ID NO: 19 - DNA, 18 bp, Artificial sequence
  Synthesized
aagacccaagtggagcag

SEQ ID NO: 20 - DNA, 17 bp, Artificial sequence
  Synthesized
tgacccagacagaaggc

SEQ ID NO: 21 - DNA, 16 bp, Artificial sequence
  Synthesized
tccttggttctgcagg

SEQ ID NO: 22 - DNA, 18 bp, Artificial sequence
  Synthesized
gcagcaggtgagacaaag

SEQ ID NO: 23 - DNA, 22 bp, Artificial sequence
  Synthesized
cascttyttagtggagagatgg

SEQ ID NO: 24 - DNA, 20 bp, Artificial sequence
  Synthesized
gtacaagcaaacagcaagtg

SEQ ID NO: 25 - DNA, 16 bp, Artificial sequence
  Synthesized
cagtccgtggaccagc

SEQ ID NO: 26 - DNA, 16 bp, Artificial sequence
  Synthesized
aacggctggagcagag

SEQ ID NO: 27 - DNA, 21 bp, Artificial sequence
  Synthesized
gcaagttaaacaaagctctcc

SEQ ID NO: 28 - DNA, 18 bp, Artificial sequence
  Synthesized
gtgcacttgccttgtagc

SEQ ID NO: 29 - DNA, 17 bp, Artificial sequence
  Synthesized
gggcatcacagggaacg

SEQ ID NO: 30 - DNA, 18 bp, Artificial sequence
  Synthesized
taccacgtggtcaagctg

SEQ ID NO: 31 - DNA, 20 bp, Artificial sequence
  Synthesized
cagtatctaggccacaatgc

SEQ ID NO: 32 - DNA, 20 bp, Artificial sequence
  Synthesized
cccaaagtcttacagatccc

SEQ ID NO: 33 - DNA, 18 bp, Artificial sequence
  Synthesized
gacggctgttttccagac

SEQ ID NO: 34 - DNA, 20 bp, Artificial sequence
  Synthesized
ggtataaacagagcgctgag

SEQ ID NO: 35 - DNA, 17 bp, Artificial sequence
  Synthesized
ggggttgtccagtctcc

SEQ ID NO: 36 - DNA, 17 bp, Artificial sequence
  Synthesized
gctgcagtcacccaaag

SEQ ID NO: 37 - DNA, 18 bp, Artificial sequence
  Synthesized
gcagtcctacaggaaggg

SEQ ID NO: 38 - DNA, 19 bp, Artificial sequence
  Synthesized
gagttacccagacacccag

SEQ ID NO: 39 - DNA, 19 bp, Artificial sequence
  Synthesized
cctaggcacaaggtgacag

SEQ ID NO: 40 - DNA, 18 bp, Artificial sequence
  Synthesized
cgaagccaaaccaagcac

SEQ ID NO: 41 - DNA, 17 bp, Artificial sequence
  Synthesized
gattggtcaggaagggc

SEQ ID NO: 42 - DNA, 18 bp, Artificial sequence
  Synthesized
ggatggagtgtcaagctg

SEQ ID NO: 43 - DNA, 19 bp, Artificial sequence
  Synthesized
ctgcagttacacagaagcc

SEQ ID NO: 44 - DNA, 19 bp, Artificial sequence
  Synthesized
cagactccacgatacctgg

SEQ ID NO: 45 - DNA, 17 bp, Artificial sequence
  Synthesized
ggtgaaagggcaaggac

SEQ ID NO: 46 - DNA, 17 bp, Artificial sequence
  Synthesized
gctggaatgtggacagg

SEQ ID NO: 47 - DNA, 18 bp, Artificial sequence
  Synthesized
cctcctctaccaaaagcc

SEQ ID NO: 48 - DNA, 23 bp, Artificial sequence
  Synthesized
ctaacctctactggtactggcag

SEQ ID NO: 49 - DNA, 20 bp, Artificial sequence
  Synthesized
ccagaaggtagcagagaccc

SEQ ID NO: 50 - DNA, 17 bp, Artificial sequence
  Synthesized
ctccacattcctgagcc

SEQ ID NO: 51 - DNA, 16 bp, Artificial sequence
  Synthesized
actctgagcctgccct

SEQ ID NO: 52 - DNA, 16 bp, Artificial sequence
  Synthesized
gccctcctcacctgag

SEQ ID NO: 53 - DNA, 22 bp, Artificial sequence
  misc_feature (3): n is inosine
  misc_feature (5): n is inosine
  Synthesized
ggntnmaggaacaaaggagaat

SEQ ID NO: 54 - DNA, 23 bp, Artificial sequence
  Synthesized
atycgttcaaatatggaaagaaa

SEQ ID NO: 55 - DNA, 18 bp, Artificial sequence
  Synthesized
ggagaaggtccacagctc

SEQ ID NO: 56 - DNA, 18 bp, Artificial sequence
  Synthesized
caactgccaacaacaagg

SEQ ID NO: 57 - DNA, 19 bp, Artificial sequence
  Synthesized
tccttccacttgcagaaag

SEQ ID NO: 58 - DNA, 15 bp, Artificial sequence
  Synthesized
acggctggccagaag

SEQ ID NO: 59 - DNA, 21 bp, Artificial sequence
  Synthesized
cakgrcytcyytcaactgcac

SEQ ID NO: 60 - DNA, 17 bp, Artificial sequence
  Synthesized
agagccacccttgacac

SEQ ID NO: 61 - DNA, 19 bp, Artificial sequence
  Synthesized
gcttygaggctgagttcag

SEQ ID NO: 62 - DNA, 21 bp, Artificial sequence
  Synthesized
ctacactgagtgttcgagagg

SEQ ID NO: 63 - DNA, 18 bp, Artificial sequence
  Synthesized
aacaggacacaggcaaag

SEQ ID NO: 64 - DNA, 15 bp, Artificial sequence
  Synthesized
ggttccacgccactc

SEQ ID NO: 65 - DNA, 15 bp, Artificial sequence
  Synthesized
tgcaggagggggaga

SEQ ID NO: 66 - DNA, 19 bp, Artificial sequence
  Synthesized
ctctgacagtctgggaagg

SEQ ID NO: 67 - DNA, 24 bp, Artificial sequence
  Synthesized
aytctgtagtcttccagaaatcac

SEQ ID NO: 68 - DNA, 24 bp, Artificial sequence
  Synthesized
attattctctgaactttcagaagc

SEQ ID NO: 69 - DNA, 18 bp, Artificial sequence
  Synthesized
tatgaaggagcctccctg

SEQ ID NO: 70 - DNA, 17 bp, Artificial sequence
  Synthesized
caagatttcaccgcacg

SEQ ID NO: 71 - DNA, 19 bp, Artificial sequence
  Synthesized
gctgactgttcaagaggga

SEQ ID NO: 72 - DNA, 20 bp, Artificial sequence
  Synthesized
aatagtatggctttcctggc

SEQ ID NO: 73 - DNA, 19 bp, Artificial sequence
  Synthesized
gcacattgatttgggagtc

SEQ ID NO: 74 - DNA, 18 bp, Artificial sequence
  Synthesized
gtatccctggatgagctg

SEQ ID NO: 75 - DNA, 18 bp, Artificial sequence
  Synthesized
ggacaatcagactgcctc

SEQ ID NO: 76 - DNA, 18 bp, Artificial sequence
  Synthesized
gatatggggcagatggtg

SEQ ID NO: 77 - DNA, 18 bp, Artificial sequence
  Synthesized
caggtgggaaatgaagtg

SEQ ID NO: 78 - DNA, 19 bp, Artificial sequence
  Synthesized
gccagagctcatgtttctc

SEQ ID NO: 79 - DNA, 18 bp, Artificial sequence
  Synthesized
ccagcagattctcagtcc

SEQ ID NO: 80 - DNA, 19 bp, Artificial sequence
  Synthesized
gtactggtatcggcaggac

SEQ ID NO: 81 - DNA, 18 bp, Artificial sequence
  Synthesized
ggtatcagcagcccagag

SEQ ID NO: 82 - DNA, 18 bp, Artificial sequence
  Synthesized
gtgtgagccagtttcagg

SEQ ID NO: 83 - DNA, 19 bp, Artificial sequence
  Synthesized
gaagcaactctgtggtgtg

SEQ ID NO: 84 - DNA, 18 bp, Artificial sequence
  Synthesized
gaacagggaagctgacac

SEQ ID NO: 85 - DNA, 18 bp, Artificial sequence
  Synthesized
ggtaccgacaggattcag

SEQ ID NO: 86 - DNA, 18 bp, Artificial sequence
  Synthesized
gcttggtatcgtcaatcg

SEQ ID NO: 87 - DNA, 17 bp, Artificial sequence
  Synthesized
gccaggaagcagagatg

SEQ ID NO: 88 - DNA, 19 bp, Artificial sequence
  Synthesized
gcacactgccttttactgg

SEQ ID NO: 89 - DNA, 20 bp, Artificial sequence
  Synthesized
gaggtgtatccctgaaaagg

SEQ ID NO: 90 - DNA, 21 bp, Artificial sequence
  Synthesized
gtactggtatcgacaagaccc

SEQ ID NO: 91 - DNA, 19 bp, Artificial sequence
  Synthesized
ggacatctgtcaaagtggc

SEQ ID NO: 92 - DNA, 19 bp, Artificial sequence
  Synthesized
ctgttggccaggtagagtc

SEQ ID NO: 93 - DNA, 21 bp, Artificial sequence
  Synthesized
gggtagccttttgtttgtttg